(19) United States
(12) Patent Application Publication
(10) Pub. No.: US 2015/0297403 A1
(43) Pub. Date: Oct. 22, 2015

(54) SYSTEM AND METHOD FOR RESECTING CORNEAL TISSUE

(71) Applicant: AMO DEVELOPMENT, LLC, Santa Ana, CA (US)

(72) Inventors: Ronald M. Kurtz, Irvine, CA (US); Tibor Juhasz, Irvine, CA (US); Melvin A. Sarayba, Ladera Ranch, CA (US); Roger Steinert, Laguna Beach, CA (US)

(73) Assignee: AMO DEVELOPMENT, LLC, Santa Ana, CA (US)

(21) Appl. No.: 14/791,093

(22) Filed: Jul. 2, 2015

Related U.S. Application Data
(63) Continuation of application No. 11/469,899, filed on Sep. 5, 2006.

(51) Int. Cl. A61F 9/008; A61F 2/14; A61F 9/009

(52) U.S. Cl. CPC A61F 9/0081; A61F 9/009; A61F 2/142; A61F 2009/00851; A61F 2009/00872

(57) ABSTRACT

A system and method of resecting corneal tissue for transplantation is disclosed. In each of the recipient cornea and the donor cornea, an annular incision is made at a predetermined incision depth. A first sidecut incision is made in each cornea, running from the outer periphery of the annular incision to one of the anterior corneal surface or the posterior corneal surface. The first sidecut incision forms an acute angle with the annular incision. A second sidecut incision is also made in each cornea, running from the inner periphery of the annular incision to the other of the anterior corneal surface or the posterior corneal surface. The second sidecut incision forms an acute angle with the annular incision. The combination of the incisions in each cornea resects corneal tissue from the recipient cornea and donor tissue from the donor cornea. The donor tissue is grafted into the recipient cornea.

### CROSS-REFERENCE TO RELATED APPLICATIONS

This application is a continuation of U.S. patent application Ser. No. 11/469,899, filed Sep. 5, 2006, hereby incorporated by reference in its entirety.

### FIELD OF THE INVENTION

The field of the present invention is systems and procedures for transplanting corneas.

### BACKGROUND

A variety of techniques presently exist for performing corneal transplants. The tools used for the different techniques range from the traditional trephine to the more advanced laser surgical systems. Regardless of the technique or tool used for the transplant procedure, the overarching goal remains to provide the recipient with new corneal tissue while minimizing post-surgical complications such as induced astigmatism. Other desirable aspects of recent improvements include reducing the overall healing time and the likelihood of additional complications from wound ruptures.

One technique which attempts to use advanced laser surgical systems to help reduce post-surgical complications is disclosed in U.S. Pat. No. 6,805,694. This technique relies on the theory that the inner and outer diameters of the undercut region should be chosen to have a specific ratio. Further, the theory postulates that the internal pressure of the eye will provide enhanced wound stability and reduce post-surgical complications by selection of an appropriate ratio. Unfortunately, when applied in actual transplant procedures, this theory does not rise up to meet expectations.

### SUMMARY OF THE INVENTION

The present invention is directed toward a system and method for resecting corneal tissue as part of a transplant procedure. In the system, a surgical laser emits a pulsed laser beam which is directed into the cornea by a focusing assembly. An interface provides a plurality of incision patterns for selection of a sidecut pattern which includes an annular region. The selected sidecut pattern is received by a controller which employs the focusing assembly to move the focal point of the pulsed laser beam and incise a donor cornea according to the sidecut pattern, placing the incision corresponding to the annular region at a predetermined depth from the anterior corneal surface. The controller also employs the focusing assembly to move the focal point and incise a recipient cornea according to the sidecut pattern, again placing the incision corresponding to the annular region at the same predetermined depth from the anterior corneal surface.

In the method, the depth of an incision from the anterior corneal surface is initially determined. This incision depth is used for both the recipient cornea and the donor cornea, and in each cornea, an annular incision is made at the incision depth. A first sidecut incision is made running from the outer periphery of the annular incision toward one of the anterior corneal surface or the posterior corneal surface. A second sidecut incision is made running from the inner periphery of the annular incision toward the other of the anterior corneal surface or the posterior corneal surface. The angle of each sidecut incision to the annular incision may be acute, perpendicular, or obtuse. The combination of the incisions in each cornea resects corneal tissue from the recipient cornea and donor tissue from the donor cornea, respectively. The donor tissue is thereafter grafted into the recipient cornea. Following the graft, the donor tissue may be secured using a plurality of sutures, each preferably passing through the sidecut incision which is nearest the posterior surface of the recipient cornea.

The extend of the sidecut incisions depends upon whether the transplant is a full thickness corneal transplant or a partial thickness corneal transplant. In a full thickness transplant, each sidecut incision runs from the annular incision to one of the anterior corneal surface or the posterior corneal surface. A partial thickness transplant may be an anterior lamellar keratoplasty (ALK) or a posterior lamellar keratoplasty (PLK). In both procedures, a resection incision is made within the stromal tissue of each cornea. For the ALK procedure, the depth of the annular incision is between the anterior surface and the resection incision, whereas for the PLK procedure, the depth of the annular incision is between the posterior surface and the resection incision. Thus, in the ALK procedure, one of the sidecut incisions runs from the annular incision to the anterior surface and the other runs from the annular incision to the resection incision. Similarly, in the PLK procedure, one of the sidecut incisions runs from the annular incision to the posterior surface and the other runs from the annular incision to the resection incision.

Accordingly, an improved system and method for resecting corneal tissue as part of a transplant procedure are disclosed. Advantages of the improvements will appear from the drawings and the description of the preferred embodiment.

### BRIEF DESCRIPTION OF THE DRAWINGS

In the drawings, wherein like reference numerals refer to similar components:

FIGS. 1A & 1B illustrates a sectional view of a cornea with incisions made therein for a full thickness corneal tissue transplant;

FIG. 2 illustrates a sectional view of donor tissue grafted into a recipient cornea;

FIG. 3 illustrates a sectional view of incisions made in a cornea for a partial thickness ALK corneal tissue transplant;

FIG. 4 illustrates a sectional view of incisions made in a cornea for a partial thickness PLK corneal tissue transplant; and

FIG. 5 is a schematic view of a system for resecting corneal tissue.

### DETAILED DESCRIPTION

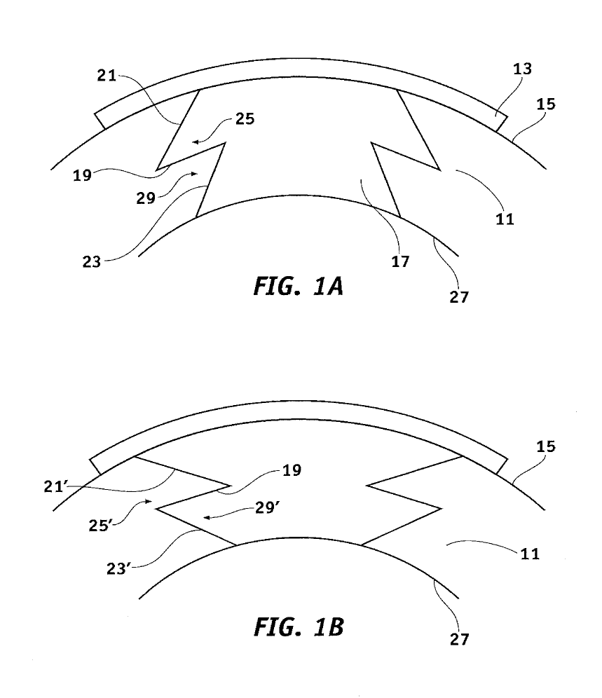

Turning in detail to the drawings, FIG. 1A illustrates the incisions made in a cornea 11 as part of a full thickness corneal transplant procedure. The same incisions are made in both the recipient cornea and the donor cornea, although the incisions in the donor cornea may be made approximately 1%-5% larger, or more preferably 2%-5% larger, than the incisions in the recipient cornea to account for shrinkage in the donor corneal tissue following resection. As part of the procedure, a contact lens 13 placed against the anterior corneal surface 15. This contact lens 13 deforms the cornea 11, forcing the anterior corneal surface 15 to take on the shape of the contact lens 13. Deformation of the cornea 11 in this manner provides multiple advantages which are well known to skilled artisans. For example, U.S. Pat. No. 5,549,632, which is incorporated herein by reference, describes advantages gained in making laser incisions by deforming the shape of the cornea, particularly by applanation. U.S. Pat. No. 6,863,667 and U.S. patent application Ser. No. 11/258,399, both of which are incorporated herein by reference, describe patient interface devices which may be used to align the surgical laser with the recipient cornea for purposes of making accurate incisions.

Further, deformation of the cornea reduces the amount of physical data which needs to be collected for both the recipient cornea and the donor cornea prior to the transplant procedure. The physical data collected includes thickness measurements of both the recipient and donor corneas. These thickness measurements are used to develop a thickness profile of each cornea. Additional physical data may also be collected for each cornea. This thickness profile, along with any other data needed for the procedure, may be obtained by any one of the many known methods for measuring the physical structure of the eye, with the preferred method being through optical coherence tomography (OCT). Many commercially available OCT scanners are capable of performing such measurements. One example is the Visante™ OCT scanning system, manufactured by Carl Zeiss Meditec, which has an office in Dublin, Calif. One advantage of the Visante™ OCT system is that it does not make contact with the cornea when performing the OCT scan.

Three incisions, an annular incision 19 and two sidecut incisions 21, 23, are made in the cornea 11 to resect the corneal tissue 17. These incisions may be made separately, one at a time, or they may be made concurrently. The combined incisions result in corneal tissue being resected from the recipient cornea, and donor tissue being resected from the donor cornea. All incisions are preferably made using a pulsed laser beam having ultra-short pulses, preferably in the femtosecond range. The laser may be of the type described in U.S. Pat. No. 4,764,930, producing an ultra-short pulsed beam as described in one or both of U.S. Pat. No. 5,984,916 and U.S. Pat. No. RE37,585. The disclosures of the aforementioned patents are incorporated herein by reference in their entirety. Commercial lasers capable of performing the incisions are available from IntraLase Corp. of Irvine, Calif.

The annular incision 19 is located at a predetermined depth from the anterior corneal surface 15. To facilitate the resection and grafting process, the entire annular incision 19 is at a uniform distance from the anterior corneal surface 15, although such uniformity of depth is not required. Further, for simplicity the annular incision 19 is described as being defined by an inner radius and an outer radius, although such is not necessary. It is sufficient for this incision to be formed by an inner perimeter and an outer perimeter, both perimeters being of any desired shape. More complex shapes, however, can add significantly to the complexity of the procedure. Various techniques are known for making the annular incision 19 at a uniform distance from the anterior corneal surface 15. For example, the techniques disclosed in U.S. Pat. No. 5,993,438, U.S. Pat. No. 6,730,074, and U.S. Patent Publication No. 20050245915 may be readily adapted to make the desired annular incision 19. Other techniques known to skilled artisans may also be employed.

The first sidecut incision 21 runs from the outer periphery of the annular incision 19 to the anterior corneal surface 15. An acute angle is formed at the juncture 25 of the first sidecut incision 21 and the annular incision 19. The second sidecut incision 23 runs from the inner periphery of the annular incision 19 to the posterior corneal surface 27. An acute angle is also formed at the juncture 29 of the second sidecut incision 23 and the annular incision 19. Alternatively, the angle between each of the sidecut incisions and the annular incision may be perpendicular or obtuse.

FIG. 1B illustrates an alternative configuration for the sidecut incisions 21′, 23′ made in the cornea 11 with respect to the annular incision 19. Here, the first sidecut incision 21′ runs from the inner perimeter of the annular incision 19 to the anterior corneal surface 15, and the second sidecut incision 23′ runs from the outer perimeter of the annular incision 19 to the posterior corneal surface 27.

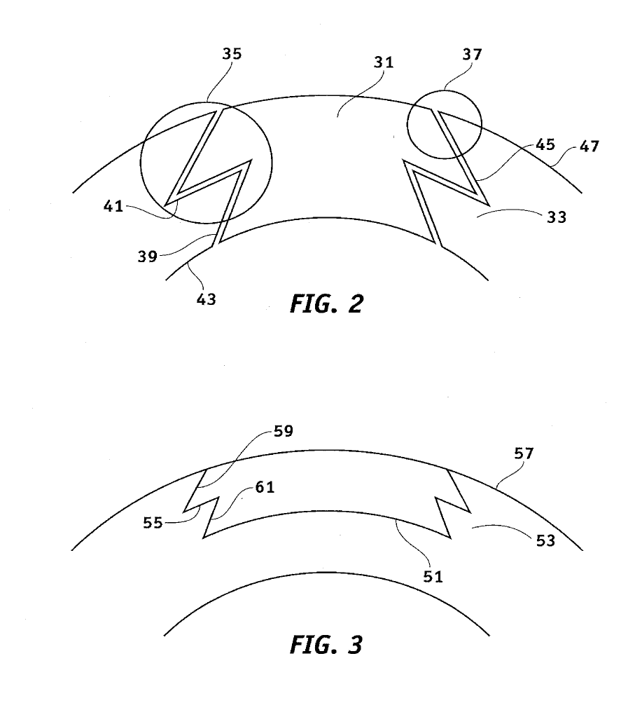

The donor tissue 31 is shown grafted into the recipient cornea 33 in FIG. 2. Sutures 35, 37 are placed to secure the donor tissue 31 to the recipient cornea 33. Two different techniques for placement of sutures are shown. The first suture 35 is placed through the sidecut incision 39 which runs between the annular incision 41 and the posterior corneal surface 43. The second suture 37 is placed through the sidecut incision 45 which runs between the annular incision 41 and the anterior corneal surface 47. Both locations of sutures are believed to be equally as effective for securing the donor tissue 31 to the recipient cornea 33. Further, neither location is believed to be more likely than the other to induce astigmatism during the healing process.

FIG. 3 illustrates how the incision configuration described above may be adapted for an ALK procedure. For the ALK process, a resection incision 51 is made in the cornea 53 at a predetermined resection depth, and the annular incision 55 is made at a depth which lies between the resection depth and the anterior corneal surface 57. The first sidecut incision 59 is made in the same manner as is described above. The second sidecut incision 61, however, runs from the annular incision to the resection incision 51. The donor tissue may be secured to the recipient cornea by either of the techniques described above

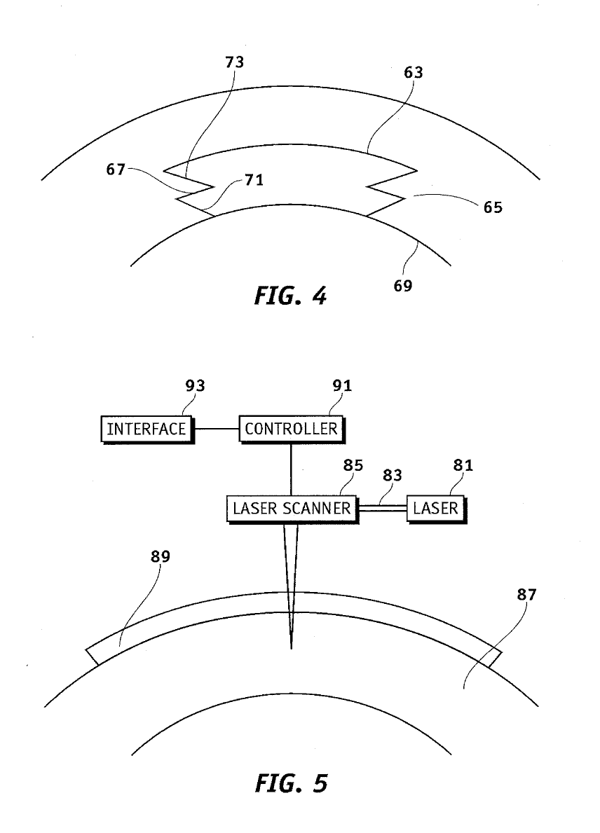

FIG. 4 illustrates how the incision configuration described above may be adapted for an PLK procedure. For the PLK process, a resection incision 63 is made in the cornea 65 at a predetermined resection depth, and the annular incision 67 is made at a depth which lies between the resection depth and the posterior corneal surface 69. The first sidecut incision 71 is made in the same manner as is described above. The second sidecut incision 73, however, runs from the annular incision to the resection incision 63. In a PLK procedure, sutures are not necessary to secure the donor tissue to the recipient cornea.

Referring to FIG. 5, a surgical system is shown which may be used to incise both a donor cornea or a recipient cornea. Alternatively, two similarly configured systems may be used to incise each respective cornea. A femtosecond surgical laser 81 generates a pulsed laser beam 83 and directs that beam into the focusing assembly 85, which in turn focuses the pulsed beam 83 into the cornea 87. A contact lens 89 is placed over the cornea to deform the anterior corneal surface as described above. Where different contact lenses are used with the donor cornea and the recipient cornea, the posterior curvature, i.e., the side of the contact lens that is placed against the anterior corneal surface, is the same for each contact lens. The controller 91 is a programmable computer which precisely controls the location of the beam focal point within the cornea 87 according to parameters received from the surgeon interface 93. The interface 93 presents the surgeon with several incision patterns from which the desired sidecut pattern is selected. The selected pattern is received by the controller 91, which uses the pattern to incise both the donor cornea and the recipient cornea, placing the annular region of the selected pattern at a predetermined depth from each respective anterior corneal surface. The contact lenses which are used to deform each of the corneas as part of this procedure preferably have the same curvature on the posterior surface, i.e., the surface placed in contact with the anterior corneal surface.

Thus, a method of transplanting corneal tissue is disclosed. While embodiments of this invention have been shown and described, it will be apparent to those skilled in the art that many more modifications are possible without departing from the inventive concepts herein. The invention, therefore, is not to be restricted except in the spirit of the following claims.

Accordingly, it is to be understood that the foregoing embodiments are merely illustrative of the invention and that no limitations are intended to either the details of the construction or design other than as defined in the appended claims.

What is claimed is:

1. A method of transplanting donor tissue from a donor cornea to a recipient cornea, the method comprising:
placing a contact lens against an anterior corneal surface of both the recipient and donor corneas to deform the cornea and take on the shape of the contact lens;
measuring a thickness of both the deformed recipient and donor corneas using optical coherence tomography (OCT) and developing a thickness profile of each cornea from the anterior corneal surface to a posterior corneal surface, respectively;
determining an incision depth beneath the anterior corneal surface of the recipient cornea;
making an annular incision using a surgical laser at the incision depth in each of the donor cornea and the recipient cornea, each annular incision having an outer periphery and an inner periphery and extending at a uniform distance from the anterior corneal surface;
making a first annular sidecut incision using a surgical laser in each of the donor cornea and the recipient cornea, the first annular sidecut incision running from either the outer periphery or the inner periphery of the annular incision to the anterior corneal surface and being angled such that the intersection of the first annular sidecut incision and the annular incision forms an acute angle;
making a second annular sidecut incision using a surgical laser in each of the donor cornea and the recipient cornea, the second annular sidecut incision running from the other of the outer periphery or inner periphery of the annular incision to the posterior corneal surface and being angled such that the intersection of the second annular sidecut incision and the annular incision forms an acute angle,
wherein a combination of the incisions in each of the recipient cornea and the donor cornea resects corneal tissue from the recipient cornea and resects the donor tissue from the donor cornea, respectively;
removing the resected recipient tissue from the recipient cornea to form a space; and
grafting the resected donor tissue into the space in the recipient cornea, wherein the incised surfaces of the resected donor tissue match the shape of the space in the recipient cornea.

2. The method of claim 1 further comprising securing the donor tissue to the recipient cornea with a plurality of sutures, at least one of the sutures passing through the second sidecut incision.

3. The method of claim 1 wherein engagement of incised surfaces of the donor tissue with incised surfaces of the recipient cornea secure the donor cornea into the recipient cornea sufficiently that sutures are not used.

4. The method of claim 1 wherein the annular, first annular sidecut, and second annular sidecut incisions made to the donor cornea are each 1-5% larger than the same respective incisions made to the recipient cornea.

5. The method of claim 1 wherein the annular, first annular sidecut, and second annular sidecut incisions made to the donor cornea are each 2-5% larger than the same respective incisions made to the recipient cornea.

6. The method of claim 1 wherein the first and second annular sidecut incisions are made one at a time.

7. The method of claim 1 wherein the first and second annular sidecut incisions are made concurrently with the annular incision.

8. A method of transplanting donor tissue from a donor cornea to a recipient cornea, the method comprising:
placing a contact lens against an anterior corneal surface of both the recipient and donor corneas to deform the cornea and take on the shape of the contact lens;
measuring a thickness of both the deformed recipient and donor corneas using optical coherence tomography (OCT) and developing a thickness profile of each cornea from the anterior corneal surface to a posterior corneal surface, respectively;
determining a first incision depth beneath the anterior corneal surface of the recipient cornea;
determining a second incision depth beneath the anterior corneal surface of the recipient cornea between the first incision depth and a posterior corneal surface such that the second incision depth is greater than the first incision depth;
making an annular incision using a surgical laser at the first incision depth in each of the donor cornea and the recipient cornea, each annular incision having an outer periphery and an inner periphery and extending at a uniform distance from the anterior corneal surface;
making a partially spherical resection incision using a surgical laser at the second incision depth in each of the donor cornea and the recipient cornea, the resection incision extending at a uniform distance from the anterior corneal surface;
making a first annular sidecut incision using a surgical laser in each of the donor cornea and the recipient cornea, the first annular sidecut incision running from either the outer periphery or the inner periphery of the annular incision to the anterior corneal surface and being angled such that the intersection of the first annular sidecut incision and the annular incision forms an acute angle;
making a second annular sidecut incision using a surgical laser in each of the donor cornea and the recipient cornea, the second annular sidecut incision running from the other of the outer periphery or inner periphery of the annular incision to an outer edge of the resection incision and being angled such that the intersection of the second annular sidecut incision and the annular incision forms an acute angle,
wherein a combination of the incisions in each of the recipient cornea and the donor cornea resects corneal tissue from the recipient cornea and resects the donor tissue from the donor cornea, respectively;
removing the resected recipient tissue from the recipient cornea to form a space; and
grafting the resected donor tissue into the space in the recipient cornea, wherein the incised surfaces of the resected donor tissue match the shape of the space in the recipient cornea.

9. The method of claim 8 wherein engagement of incised surfaces of the donor tissue with incised surfaces of the recipient cornea secure the donor cornea into the recipient cornea sufficiently that sutures are not used.

10. The method of claim 8 wherein the annular, first annular sidecut, and second annular sidecut incisions made to the donor cornea are each 1-5% larger than the same respective incisions made to the recipient cornea.

11. The method of claim 8 wherein the annular, first annular sidecut, and second annular sidecut incisions made to the donor cornea are each 2-5% larger than the same respective incisions made to the recipient cornea.

12. The method of claim 8 wherein the first and second annular sidecut incisions are made one at a time.

13. The method of claim 8 wherein the first and second annular sidecut incisions are made concurrently with the annular incision.

14. A method of transplanting donor tissue from a donor cornea to a recipient cornea, the method comprising:
placing a contact lens against an anterior corneal surface of both the recipient and donor corneas to deform the cornea and take on the shape of the contact lens;
measuring a thickness of both the deformed recipient and donor corneas using optical coherence tomography (OCT) and developing a thickness profile of each cornea from the anterior corneal surface to a posterior corneal surface, respectively;
determining a first incision depth beneath the anterior corneal surface of the recipient cornea;
determining a second incision depth beneath the anterior corneal surface of the recipient cornea between the first incision depth and a posterior corneal surface such that the second incision depth is greater than the first incision depth;
making a partially spherical resection incision using a surgical laser at the first incision depth in each of the donor cornea and the recipient cornea, the resection incision extending at a uniform distance from the anterior corneal surface;
making an annular incision using a surgical laser at the second incision depth in each of the donor cornea and the recipient cornea, each annular incision having an outer periphery and an inner periphery and extending at a uniform distance from the anterior corneal surface;
making a first annular sidecut incision using a surgical laser in each of the donor cornea and the recipient cornea, the first annular sidecut incision running from either the outer periphery or the inner periphery of the annular incision to an outer edge of the resection incision and being angled such that the intersection of the first annular sidecut incision and the annular incision forms an acute angle;
making a second annular sidecut incision using a surgical laser in each of the donor cornea and the recipient cornea, the second annular sidecut incision running from the other of the outer periphery or inner periphery of the annular incision to the posterior corneal surface and being angled such that the intersection of the second annular sidecut incision and the annular incision forms an acute angle,
wherein a combination of the incisions in each of the recipient cornea and the donor cornea resects corneal tissue from the recipient cornea and resects the donor tissue from the donor cornea, respectively;
removing the resected recipient tissue from the recipient cornea to form a space; and
grafting the resected donor tissue into the space in the recipient cornea, wherein the incised surfaces of the resected donor tissue match the shape of the space in the recipient cornea.

15. The method of claim 14 wherein engagement of incised surfaces of the donor tissue with incised surfaces of the recipient cornea secure the donor cornea into the recipient cornea sufficiently that sutures are not used.

16. The method of claim 14 wherein the annular, first annular sidecut, and second annular sidecut incisions made to the donor cornea are each 1-5% larger than the same respective incisions made to the recipient cornea.

17. The method of claim 8 wherein the annular, first annular sidecut, and second annular sidecut incisions made to the donor cornea are each 2-5% larger than the same respective incisions made to the recipient cornea.

18. The method of claim 14 wherein the first and second annular sidecut incisions are made one at a time.

19. The method of claim 14 wherein the first and second annular sidecut incisions are made concurrently with the annular incision.
